(19) United States
(12) Patent Application Publication
(10) Pub. No.: US 2016/0287627 A1
(43) Pub. Date: Oct. 6, 2016

(54) METHOD FOR SUPPRESSING ONSET OF GASTRIC ULCER AS ADVERSE EFFECT OF DRUG, ORAL PHARMACEUTICAL COMPOSITION FOR SUPPRESSING ONSET OF GASTRIC ULCER AND METHOD FOR PRODUCING THE SAME

(71) Applicants: KEWPIE CORPORATION, Shibuya-ku Tokyo (JP); TECHNO GUARD CO., LTD., Kawasaki-shi Kanagawa (JP)

(72) Inventors: Shuta FURUKI, Chofu-shi, Tokyo (JP); Hideto YOSHIDA, Chofu-shi, Tokyo (JP); Kiichiro NABETA, Kawasaki-shi, Kanagawa (JP)

(73) Assignees: KEWPIE CORPORATION, Shibuya-ku, Tokyo (JP); TECHNO GUARD CO., LTD., Kawasaki-shi, Kanagawa (JP)

(21) Appl. No.: 15/037,404

(22) PCT Filed: Nov. 19, 2014
(86) PCT No.: PCT/JP2014/080626

§ 371 Date: May 18, 2016

(30) Foreign Application Priority Data
Nov. 22, 2013 (JP) 2013-242082

(51) Int. Cl. A61K 31/728; A61K 9/06; A61K 31/405; A61K 31/192; A61K 31/616; A61K 9/00; A61K 31/196

(52) U.S. Cl. CPC A61K 31/728; A61K 9/0053; A61K 9/06; A61K 31/196; A61K 31/192; A61K 31/616; A61K 31/405

(57) ABSTRACT

[Problem] To suppress the onset of adverse effects of drugs that cause gastric ulcers as an adverse effect.

[Solution] The onset of gastric ulcers induced by a drug is suppressed by incorporating, into a pharmaceutical composition containing a drug having gastric ulcers as an adverse effect, a high-molecular polysaccharide that has a molecular weight of 800,000-3,000,000 and that produces a gel when a 0.3% aqueous solution thereof is added dropwise to artificial gastric juice.

### TECHNICAL FIELD

The present invention relates to, in an oral pharmaceutical composition of a drug having gastric ulcers as an adverse effect, a method for suppressing onset of gastric ulcer induced by the drug, and an oral pharmaceutical composition with suppressed onset of gastric ulcer and a method for producing the same. In details, it relates to a method for suppressing onset of gastric ulcer by incorporating a high-molecular polysaccharide that meets certain requirements into the pharmaceutical composition containing a drug having gastric ulcers as an adverse effect, and an oral pharmaceutical composition with suppressed onset of gastric ulcer and a method for producing the same.

### BACKGROUND OF THE INVENTION

Among anti-inflammatory drugs (NSAIDs), hormone preparations, anticancer agents, therapeutic agents of osteoporosis, antiplatelet drugs, and the like that are currently used widely, many drugs are known to have gastric ulcers as an adverse effect. Thus, in case that such drugs having gastric ulcers as an adverse effect are administered orally, it is necessary to suppress onset of gastric ulcer by administering in combination with a preventive agent for gastric ulcer such as Mucosta Tablets. However, from the viewpoint of patient medication compliance, it is desirable that a drug is able to be administered as a preparation with suppressed onset of gastric ulcer as an adverse effect of the drug, thereby the administration of a preventive agent for gastric ulcer can be avoided.

Meanwhile, there are many reports wherein high-molecular polysaccharides are applied to a prevention and treatment for gastrointestinal diseases including ulcer. For example, patent document 1 discloses that by oral administration of zinc hyaluronate, the wall and mucosa of gastrointestinal tract is protected and gastrointestinal ulcer is prevented and treated, wherein zinc hyaluronate itself is used as main medicine. In addition, patent document 2 discloses therapeutic and preventive drugs for gastritis and duodenal disease which are a combination of hyaluronic acid, chondroitin sulfate and aluminum hydroxide. Patent document 3 discloses prevention and treatment of gastrointestinal diseases (including ulcer) by bismuth hyaluronate. Furthermore, patent document 4 discloses therapeutic drugs for peptic ulcer by a mixture of low-molecular hyaluronic acid and high-molecular hyaluronic acid. However, in these documents, high-molecular polysaccharides work as a main medicine respectively. In contrast, in patent document 5, a preparation is disclosed wherein fucoidan that is a sulfated polysaccharide is compounded into the ingredients of anti-inflammatory agent in order to reduce adverse effects such as drug-induced gastrointestinal disease caused by nonsteroidal anti-inflammatory agents.

### PRIOR ART DOCUMENTS

### Patent Documents

Patent document 1: U.S. Pat. No. 6,656,921 B1

Patent document 2: WO 2010/136872 A1

Patent document 3: U.S. Pat. No. 8,093,374 B1

Patent document 4: JP 2011-37849 A

Patent document 5: JP H11-263730 A

### SUMMARY OF INVENTION

### Problems to be Resolved by the Invention

Under the above-described circumstances, as the result of extensive study on a method for suppressing adverse effects of drugs having onset of gastric ulcer as the adverse effect of the drugs, the present inventors have found that an oral pharmaceutical composition with suppressed onset of gastric ulcer can be obtained by compounding and formulating certain high-molecular polysaccharides in drugs having onset of gastric ulcer as an adverse effect, and completed the present invention. Thereby, for the drugs that are conventionally needed to suppress the onset of gastric ulcer by additional administration of preventive agent for gastric ulcer, the administration of the preventive agent for gastric ulcer is not needed. The patient medication compliance is improved. In addition, the reduction of the patient burdens can be possible in terms of costs.

### Means of Solving the Problems

That is, the present invention provides;
(1) A method for suppressing onset of gastric ulcer caused by a drug having an adverse effect of gastric ulcer, by using said drug in combination with a high-molecular polysaccharide having molecular weight of not less than 800,000 and not more than 3,000,000, which produces a gel when a 0.3% (w/v) aqueous solution thereof is added dropwise to artificial gastric juice.
(2) A method for suppressing onset of gastric ulcer caused by a drug having an adverse effect of gastric ulcer, by incorporating a high-molecular polysaccharide having molecular weight of not less than 800,000 and not more than 3,000,000, which produces a gel when a 0.3% (w/v) aqueous solution thereof is added dropwise to artificial gastric juice into a pharmaceutical composition containing said drug.
(3) The method for suppressing onset of gastric ulcer according to the above-mentioned (1) or (2), wherein the drug having an adverse effect of gastric ulcer is an anti-inflammatory drug (NSAIDs), a hormone preparation, an anticancer agent, a therapeutic agent of osteoporosis or an antiplatelet drug.
(4) The method for suppressing onset of gastric ulcer according to any one of the above-mentioned (1) to (3), wherein the high-molecular polysaccharide is hyaluronic acid and/or a salt thereof, or Xanthan gum.
(5) The method for suppressing onset of gastric ulcer according to any one of 1 to 4, wherein the onset of gastric ulcer is suppressed without changing the approved administration and dosage of the drug having an adverse effect of gastric ulcer.
(6) An oral pharmaceutical composition with suppressed onset of gastric ulcer which contains
a drug having an adverse effect of gastric ulcer,
a high-molecular polysaccharide having molecular weight of not less than 800,000 and not more than 3,000,000, which produces a gel when a 0.3% (w/v) aqueous solution thereof is added dropwise to artificial gastric juice, and a pharmaceutically acceptable carrier.
(7) The oral pharmaceutical composition according to the above-mentioned (6), wherein the drug having an adverse effect of gastric ulcer is an anti-inflammatory drug (NSAIDs), a hormone preparation, an anticancer agent, a therapeutic agent of osteoporosis or an antiplatelet drug.
(8) The oral pharmaceutical composition according to the above-mentioned (6) or (7), wherein the high-molecular polysaccharide is hyaluronic acid and/or a salt thereof, or Xanthan gum, and
(9) A method for producing a pharmaceutical preparation with suppressed onset of gastric ulcer as an adverse effect, characterized by mixing previously a high-molecular polysaccharide having molecular weight of not less than 800,000 and not more than 3,000,000 which produces a gel when a 0.3% (w/v) aqueous solution thereof is added dropwise to artificial gastric juice, in a drug having an adverse effect of gastric ulcer, then formulating the mixture into a preparation.

### Effect of the Invention

According to the present invention, oral pharmaceutical compositions with suppressed onset of gastric ulcer can be obtained by compounding certain high-molecular polysaccharides in a drug having onset of gastric ulcer as an adverse effect and formulating. Therefore, for the drugs that are conventionally needed to suppress the onset of gastric ulcer by additional administration of preventive agent for gastric ulcer, the administration of the preventive agent for gastric ulcer is not needed. The patient medication compliance is improved. In addition, the patient burdens can be reduced in terms of costs.

### BRIEF DESCRIPTION OF THE DRAWINGS

FIG. 1 is a graph showing the relation between various high-molecular polysaccharides and onset suppression of gastric ulcer in Test Example 1.

FIG. 2 is a graph showing the relation between molecular weight of hyaluronic acid and onset suppression of gastric ulcer in Test Example 2.

FIG. 3 is a graph showing the relation between the compounded amount of hyaluronic acid and onset suppression of gastric ulcer in Test Example 3.

FIG. 4 includes pictures showing appearance of gelation of various high-molecular polysaccharides in artificial gastric juice.

### MODE FOR CARRYING OUT THE INVENTION

The present invention relates to a method for suppressing onset of gastric ulcer caused by a drug having an adverse effect of gastric ulcer, by using said drug having an adverse effect of gastric ulcer in combination with a high-molecular polysaccharide having molecular weight of not less than 800,000 and not more than 3,000,000 which produces a gelled substance when a 0.3% (w/v) aqueous solution thereof is added dropwise to artificial gastric juice, and a method for suppressing onset of gastric ulcer caused by a drug having an adverse effect of gastric ulcer, by incorporating a high-molecular polysaccharide having molecular weight of not less than 800,000 and not more than 3,000,000 which produces a gelled substance when a 0.3% (w/v) aqueous solution thereof is added dropwise to artificial gastric juice into a pharmaceutical composition containing said drug having an adverse effect of gastric ulcer, and an oral pharmaceutical composition with suppressed onset of gastric ulcer which contains a drug having an adverse effect of gastric ulcer, and a high-molecular polysaccharide having molecular weight of not less than 800,000 and not more than 3,000,000 which produces a gelled substance when a 0.3% (w/v) aqueous solution thereof is added dropwise to artificial gastric juice, and a pharmaceutically acceptable carrier, and a method for producing the same. That is, the present invention relates to onset suppression of gastric ulcer caused by oral administration of the drug, by incorporating the HA of the present invention into the drug having an adverse effect of onset of gastric ulcer when administered orally alone in a therapeutically effective amount.

Herein, the “drug having an adverse effect of gastric ulcer” includes anti-inflammatory drugs (NSAIDs), hormone preparations, anticancer agents, and therapeutic agents of osteoporosis or antiplatelet drugs. Specifically, examples of anti-inflammatory drugs (NSAIDs) include loxoprofen sodium, zaltoprofen, pranoprofen, ibuprofen, ketoprofen, flurbiprofen, flurbiprofen axetil, oxaprozin, tiaprofenic acid, naproxen, fenoprofen calcium, alminoprofen, indomethacin, indomethacin farnesil, diclofenac sodium, sulindac, fenbufen, acemetacin, amfenac sodium, nabumetone, proglumetacin maleate, etodolac, mofezolac, ampiroxicam, piroxicam, tenoxicam, meloxicam, lornoxicam, sodium salicylate, acetylsalicylate, salicylamide, flufenamic acid aluminium, mefenamic acid, tolfenamic acid, bucolome, meloxicam, nabumetone, etodolac, tiaramide hydrochloride, epirizole, and emorfazone. Examples of hormone preparation include anastrozole, exemestane, estramustine, ethinyl estradiol, chlormadinone, goserelin, tamoxifen, dexamethasone, toremifene, bicalutamide, flutamide, prednisolone, fosfestrol, mitotane, methyltestosterone, medroxyprogesterone, mepitiostane, leuprorelin, and letrozole. Examples of anticancer agents include ifosfamide, cyclophosphamide, dacarbazine, temozolomide, nimustine, busulfan, melphalan, enocitabine, capecitabine, carmofur, gemcitabine, cytarabine, tegafur, tegafur uracil, nelarabine, fluorouracil, fludarabine, pemetrexed, pentostatin, methotrexate, irinotecan, etoposide, sobuzoxane, docetaxel, nogitecan, paclitaxel, vinorelbine, vincristine, vindesine, vinblastine, actinomycin D, aclarubicin, idarubicin, epirubicin, daunorubicin, doxorubicin, pirarubicin, bleomycin, peplomycin, mitomycin C, mitoxantrone, oxaliplatin, carboplatin, cisplatin, nedaplatin, interferon-α, interferon-β, interferon-γ, interleukin2, ubenimex, freeze-dried BCG, and lentinan. Examples of therapeutic agents of osteoporosis include etidronic acid, alendronic acid, risedronic acid, minodronic acid, ipriflavone, and estradiol. Additionally, examples of antiplatelet drugs include acetylsalicylic acid.

The “high-molecular polysaccharide” is not particularly limited so long as it has the characteristics of being a high-molecular polysaccharide having molecular weight of not less than 800,000 and not more than 3,000,000 and producing gelation product when a 0.3% (w/v) aqueous solution thereof is added dropwise to artificial gastric juice. Specifically, hyaluronic acid and/or a salt thereof, xanthan gum and the like are exemplified. Among them hyaluronic acid and/or a salt thereof are preferred.

In the present invention, the “hyaluronic acid” refers to a polysaccharide having one or more repeating constituent units consisting of disaccharide composed of glucuronic acid and N-acetyl glucosamine. In addition, the “salt of hyaluronic acid” is not particularly limited, but salts of inorganic substances known for antiulcer activity such as zinc, aluminum, bismuth and the like are excluded. Specifically, sitologically or pharmaceutically acceptable salts are preferred. For example, sodium salt, potassium salt, calcium salt, magnesium salt, ammonium salt and the like are exemplified, and sodium salt is more preferred.

Hyaluronic acids are basically those having 2 or more sugars containing at least one disaccharide unit linked via position 1 of β-D-glucuronic acid and position 3 of β-D-N-acetyl glucosamine, and basically composed of β-D-glucuronic acid and β-D-N-acetyl glucosamine, and plural disaccharide units are linked. Said sugar may be an unsaturated sugar. Examples of the unsaturated sugar include a non-reducing terminal sugar, usually, those being unsaturated between positions 4 and 5 carbons of glucuronic acid.

The hyaluronic acids and/or a salt thereof may be those extracted from natural products such as animals and the like (e.g., biological tissue such as cockscomb, umbilical cord, skin, synovial fluid, and the like), or those obtained by cultivating microorganisms, animal cells or plant cells (e.g., fermentation method using Streptococcus bacteria and the like), and those synthesized chemically or enzymatically can also be used.

As the hyaluronic acid and/or a salt thereof, any of crude extracts and purified products can be used. However, the purified products, specifically hyaluronic acid and/or a salt thereof having a purity of 90% (mass ratio) or more are preferably used.

From the viewpoint of effectively suppressing the onset of gastric ulcer, the mean molecular weight of hyaluronic acid and/or a salt thereof is preferably not less than 800,000 and not more than 3,000,000, more preferably not less than 1,000,000 and not more than 2,000,000. In addition, the mean molecular weight of hyaluronic acid and/or a salt thereof can be determined with the following method.

<Method for measuring molecular weight of hyaluronic acid>

That is, a solution wherein about 0.05 g of hyaluronic acid and/or a salt thereof is precisely weighed and dissolved in 0.2 mol/L solution of sodium chloride, followed by being adjusted precisely to 100 mL. 8 mL, 12 mL and 16 mL of said solution are taken precisely and added with the 0.2 mol/L solution of sodium chloride respectively to make precisely 20 mL. These are taken as sample solutions. Specific viscosity of the sample solutions and the 0.2 mol/L solution of sodium chloride, is measured at 30.0±0.1° C. according to The Japanese Pharmacopeia (Fourteenth Edition), General Test, Viscosity Determination (Method 1 Viscosity measurement by capillary tube viscometer) (equation (A)), and the reduced viscosity at each concentration is calculated(equation (B)). A graph is drawn with the reduced viscosity as the vertical axis and the concentration of the reduced dry sample materials (g/100 mL) as the horizontal axis. The limiting viscosity is determined from the intersection point of the straight line connecting each point and the vertical axis. The limiting viscosity obtained here is substituted into the Laurent's equation (quation (C)), and the mean molecular weight is calculated (Torvard C Laurent, Marion Ryan, and Adolph Pietruszkiewicz, “Fractionation of hyaluronic Acid”, Biochemina et Biophysica Acta., 42, 476-485(1960), Chikako Yomota, “Evaluation of Molecular Weights of Sodium Hyaluronate Preparations by SEC-MALLS”, Bull. Natl. Inst. Health Sci., 121, 030-033 (2003)).

Specific viscosity={(the required downflowing seconds of sample solution)/(the required downflowing seconds of 0.2 mol/L sodium chloride solution)}−1 (Equation A)

Reduced viscosity (dL/g)=specific viscosity/(concentration of the reduced dry sample material (g/100 mL)) (Equation B)

Limiting viscosity (dL/g)=3.6×10−4M0.78 (Equation C)

M: mean molecular weight

From the viewpoint of effectively suppressing the onset of gastric ulcer, the mean molecular weight of xanthan gum is preferably not less than 800,000 and not more than 3,000,000, more preferably not less than 1,000,000 and not more than 2,500,000.

The “produces a gel when a 0.3% (w/v) aqueous solution is added dropwise to artificial gastric juice” of the present invention means that a gelled substance is produced when a 0.3% (w/v) aqueous solution of high-molecular polysaccharide is gently added dropwise from heights within 1 cm of the liquid surface of artificial gastric juice, and it can be confirmed visually. In the present invention,anything that turns into a gel-like substance, whatever its viscosity and/or hardness, and its contour in the artificial gastric juice can be confirmed visually is called gelled substance.

From the viewpoint of effectively suppressing the onset of gastric ulcer, the gelled substance having a stronger gelation level is preferred. Since gelled substances have a tendency to take a shape of almost circular form when gelation level is strong, the shape of gelled substance is preferably almost circular form. The aqueous solution of high-molecular polysaccharide added dropwise gels while spreading spontaneously in the artificial gastric juice. Thus the almost circular form is not limited to a strict circular form, and includes also oval form and shape having a cant or protrusion in part.

In the present invention, the artificial gastric juice is prepared by the method according to the article of The Japanese Pharmacopeia, Sixteenth Edition, (first fluid for disintegration test). That is, it is prepared by adding hydrochloric acid 7.0 mL and purified water to sodium chloride 2.0 g to dissolve and making 1000 mL. Its pH is about 1.2.

From the viewpoint of effectively alleviating the adverse effect of gastric ulcer, the compounding amount of the high-molecular polysaccharide in the pharmaceutical composition of the present invention is, relative to 1 pts. mass (also referred to as pts.wt. (parts by weight)) of drug, preferably not less than 0.1 pts. mass and not more than 100 pts. mass, more preferably not less than 0.3 pts. mass and not more than 50 pts. mass, furthermore preferably not less than 0.5 pts. mass and not more than 10 pts. mass.

In addition, in the case that the pharmaceutical composition is a liquid or a capsule, from the viewpoint of effectively alleviating the adverse effect of gastric ulcer, concentration of the polysaccharide (w/v) is preferably not less than 0.05% and not more than 8%, more preferably not less than 0.25% and not more than 6%, furthermore preferably not less than 0.4% and not more than 3%. Furthermore, from the viewpoint of effectively alleviating the adverse effect of gastric ulcer, a single dose of high-molecular polysaccharides is preferably 1 g or less, more preferably not less than 5 mg and not more than 500 mg, further more preferably not less than 10 mg and not more than 200 mg.

As the pathogenic mechanism of gastric ulcer caused by a drug having an adverse effect of gastric ulcer, it is assumed that it occurs by contact of drug with the surface of stomach. Namely, at low pH, a drug takes a nonionic form, thus becomes easy to be absorbed in stomach. The drug absorbed from stomach takes ionic form because pH of solution is 4 or more, and stays there. Therefore, gastric ulcer is believed to occur easily.

Furthermore, as the mechanism of suppressing onset of gastric ulcer in the present invention, it is considered that the high-molecular polysaccharide in the pharmaceutical composition of the present invention forms gel caused by gastric juice. The gel passes through the stomach with enclosing the drug having an adverse effect of gastric ulcer. In addition, the absorption of drug in the stomach is inhibited by being enclosed with gel. And it is considered that when the drug passes through the stomach, the gel is dissolved in the intestine which is neutral, and the drug is absorbed.

The oral pharmaceutical composition of the present invention includes, for example, tablets (including sugar coated tablets, film coated tablets), pills, granules, powders, capsules (including soft capsules, microcapsules), syrups, emulsions, suspensions, and the like. As the method for producing the above-described dosage form, a known method for production used generally in the field (e.g., methods described in The Japanese Pharmacopeia, etc.) can be applied. In addition, when producing the above-described dosage form, if necessary, excipients, binding agents, disintegrating agents, lubricants, sweeteners, surfactants, suspending agents, emulsifying agents, and the like which are used conventionally in the field of preparation when formulating into the dosage form, can be appropriately contained in an appropriate amount.

For example, when tablets are produced, excipients, binding agents, disintegrating agents, lubricants and the like can be contained. When pills and granules are produced, excipients, binding agents, disintegrating agents and the like can be contained. Furthermore, when powder and capsules are produced, excipients and the like can be contained. When syrups are produced, sweeteners and the like can be contained. When emulsions and suspensions are produced, suspending agents, surfactants, emulsifying agents and the like can be contained.

Examples of the excipients include lactose, white soft sugar, glucose, starch, sucrose, microcrystalline cellulose, powdered glycyrrhiza, mannitol, sodium hydrogen carbonate, calcium phosphate, calcium sulfate and the like.

Examples of the binding agents include 5-10 wt. % starch paste solution, 10-20wt. % gum arabic solution or gelatin solution, 1-5wt. % tragacanth solution, carboxymethylcellulose solution, sodium alginate solution, glycerin and the like.

Examples of the disintegrating agents include starch, calcium carbonate and the like.

Examples of the lubricants include magnesium stearate, stearic acid, calcium stearate, purified talc and the like.

Examples of the sweeteners include glucose, fructose, invert sugar, sorbitol, xylitol, glycerin, simple syrup and the like.

Examples of the surfactants include sodium lauryl sulfate, polysorbate 80, sorbitan mono-fatty acid ester, polyoxyl stearate 40 and the like.

Examples of the suspending agents include gum arabic, sodium alginate, carboxymethylcellulose sodium, methylcellulose, bentonite and the like.

Examples of the emulsifying agents include gum arabic, tragacanth, gelatin, polysorbate 80 and the like.

Furthermore, when producing the above-described dosage form, colorants, preservatives, fragrances, flavoring agents, stabilizers, thickeners and the like which are used conventionally in the field of preparation, can be appropriately added in an appropriate amount, if desired.

In the present invention, the “approved administration and dosage” refers to the administration and dosage wherein the therapeutical effectiveness of the drug is approved by public organization.

The present invention will be specifically illustrated in reference to the following Test Examples and Examples. The present invention is not limited by these Examples in any way.

### TEST EXAMPLE 1

In Test Example 1, in order to confirm the relationship between kind of polysaccharide and the effect of reducing the adverse effect of gastric ulcer, dispersion solutions of preparation formulated with diclofenac having an adverse effect of gastric ulcer and polysaccharide were administered orally with drinking water to rats.

<Preparation of Test Formulations>

Diclofenac bulk 200 mg, hyaluronic acid (mean molecular weight 1,200,000, white powder, manufactured by Kewpie Co.) 120 mg and injection water 40 mL were placed to mix in a 100 mL Erlenmeyer flask with a stopper, and sonicated at 50° C. for 90 minutes (oscillation mode: High) with an ultrasonic cleaner (US-1R, manufactured by AS ONE Co.) to prepare Test Formulation 1.

Test Formulation 2, Test Formulation 3 and Test Formulation 4 were prepared according to the same method as that for preparing Test Formulation 1 except that the hyaluronic acid in the preparation of Test Formulation 1 was substituted with xanthan gum 200 mg, alginic acid 120 mg and gum arabic 120 mg, respectively. In addition, Control Test Formulation was prepared by excluding hyaluronic acid used in Test Formulation 1 from the combination.

<Experiment on animals>

8 week-old healthy male Slc:SD rats were purchased and bred to acclimate for 7 days by giving general solid feed. On the final day of habituation, the animals having no abnormalities in the general condition and body weight gain were selected followed by fasting for about 18 hours until the following morning. Then, animals were assigned to groups by the body weight stratified random sampling method based on the body weight on the very day of administration of the Test Formulations. After the grouping, the Test Formulations were forcedly orally administered at a single dose which was adjusted so as to be 10 mL/kg body weight of rat. At 240 minutes after the administration of the Test Formulations, the animals were euthanized by bleeding under an inhalation anesthesia of isoflurane and their stomachs were extirpated. The stomachs extirpated were subjected to formalin fixation.

Clip was removed from the formalin fixed stomach sample and incised from pylorus of stomach along greater curvature. After the incision, the stomach content was washed with physiological saline. After the washing, the part of stomach injury was observed under a stereoscopic microscope equipped with a graduated eyepiece, the area of the stomach injury (bleeding portion) was measured, and calculated the mean value thereof.

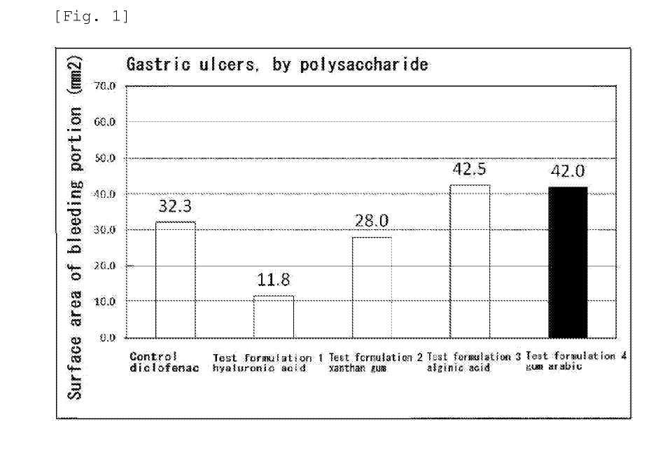

The results are shown in Table 1 and FIG. 1.

TABLE 1

| | | Mean value of |
|---|---|---|
| | | surface area of |
| | | bleeding portion |
| | polysaccharides | (mm²) |
| Control Test Formulation | None | 32.3 |
| Test Formulation 1 | hyaluronic acid | 11.8 |
| Test Formulation 2 | xanthan gum | 28 |
| Test Formulation 3 | alginic acid | 42.5 |
| Test Formulation 4 | gum arabic | 42 |

As seen from Table 1 and FIG. 1, an effect of reducing the adverse effect of gastric ulcer caused by diclofenac was observed on hyaluronic acid and xanthan gum, but the effect was not recognized for alginic acid and gum arabic.

### TEST EXAMPLE 2

In Test Example 2, in order to confirm the relationship between molecular weight of polysaccharide and the effect of reducing the adverse effect of gastric ulcer, dispersion solutions of preparation formulated with diclofenac having an adverse effect of gastric ulcer and hyaluronic acid were administered orally with drinking water to rats.

<Preparation of Test Formulations>

Test Formulation 5 and Test Formulation 6 were prepared according to the same method as that for preparing Test Formulation 1 except that the hyaluronic acid in the preparation of Test Formulation 1 was substituted with hyaluronic acid (mean molecular weight: 300,000, white powder, manufactured by Kewpie Co.) and hyaluronic acid (mean molecular weight: 8,000, white powder, manufactured by Kewpie Co.), respectively.

<Experiment on Animals>

Experiment on animals was conducted with the same method as that in Test Example 1.

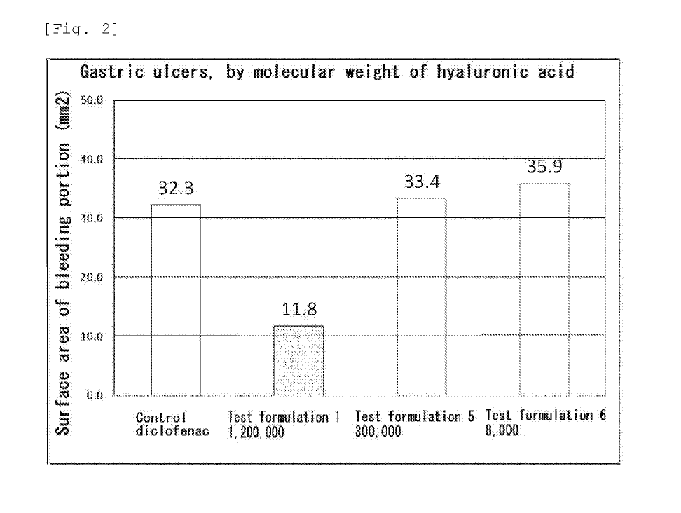

The results are shown in Table 2 and FIG. 2. The effect of reducing the adverse effect of gastric ulcer caused by diclofenac was observed on hyaluronic acid having 1,200,000 of molecular weight. However, the effect was not recognized for hyaluronic acid having 300,000 or less of molecular weight.

TABLE 2

| | | Mean value of |
|---|---|---|
| | Molecular | surface area of |
| | weight of | bleeding portion |
| | hyaluronic acid | (mm²) |
| Control Test Formulation | None | 32.3 |
| Test Formulation 1 | 1,200,000 | 11.8 |
| Test Formulation 5 | 300,000 | 33.4 |
| Test Formulation 6 | 8,000 | 35.9 |

### TEST EXAMPLE 3

In Test Example 3, in order to confirm the relationship between the compounding amount of polysaccharide and the effect of reducing the adverse effect of gastric ulcer, dispersion solutions of preparation formulated with diclofenac having an adverse effect of gastric ulcer and hyaluronic acid were administered orally with drinking water to rats.

<Preparation of Test Formulations>

Test Formulation 7 and Test Formulation 8 were prepared according to the same method as that for preparing Test Formulation 1 except that the content of hyaluronic acid 120 mg in the preparation of Test Formulation 1 was changed to 40 mg and 200 mg, respectively.

<Experiment on Animals>

Experiment on animals was conducted with the same method as that in Test Example 1.

The results are shown in Table 3 and FIG. 3.

TABLE 3

| | | Mean value of |
|---|---|---|
| | Content of | surface area of |
| | hyaluronic acid | bleeding portion |
| | (M.W. 1,200,000) | (mm²) |
| Control Test Formulation | None | 32.3 |
| Test Formulation 7 | 40 mg | 21.1 |
| Test Formulation 1 | 120 mg | 11.8 |
| Test Formulation 8 | 200 mg | 5.6 |

From the result of Test Formulation 7 in Table 3, the effect of reducing the adverse effect of gastric ulcer was observed with the combination of hyaluronic acid 0.2 pts. wt. relative to diclofenac 1 pts. wt.. Furthermore, a large increase in the effect of reducing the adverse effect was observed as the compounding amount of hyaluronic acid increases from 0.6 pts. wt. to 1 pts. wt.

### TEST EXAMPLE 4

In Test Example 4, in order to confirm the mechanism of kind of polysaccharide and effect of reducing the adverse effect of gastric ulcer, the formation of a gelled substance was confirmed by adding dropwise an aqueous solution of high-molecular polysaccharide to artificial gastric juice.

<Preparation of Artificial Gastric Juice>

The artificial gastric juice was prepared by placing purified water 500 mL and sodium chloride 2.0 g in a 1000 mL graduated cylinder, followed by mixing and dissolving, adding. hydrochloric acid 7.0 mL and mixingand then diluting with purified water to a total volume of 1000 mL.

<Preparation of Aqueous High-Molecular Polysaccharide Solutions>

Hyaluronic acid (mean molecular weight: 1,200,000, white powder, manufactured by Kewpie Co.) 90 mg and injection water 30 mL were placed in a 50 mL beaker and mixed, followed by stirring for 180 minutes with a DC stirrer (DCL-2S, manufactured by TOKYO RIKAKIKAI CO. LTD) to prepare a 0.3% aqueous solution of hyaluronic acid. 0.3% aqueous solutions of various polysaccharides were prepared according to the same method except that the hyaluronic acid in the preparation of aqueous hyaluronic acid solution was substituted with the high-molecular polysaccharide used in the above-described Test Formulations 2-4 and 6, hyaluronic acid (mean molecular weight: 800,000, manufactured by Kewpie Co.), hyaluronic acid (mean molecular weight: 1,600,000, manufactured by Kewpie Co.) and hyaluronic acid (mean molecular weight: 2,000,000, manufactured by Kewpie Co.).

<Experimental Method>

To a 50 mL beaker containing artificial gastric juice 10 mL was gently added dropwise 100 L each of aqueous solution of high-molecular polysaccharide using a micropipette from a height within 1cm from the liquid level of the artificial gastric juice. The presence or absence of gelation was visually determined.

<Result>

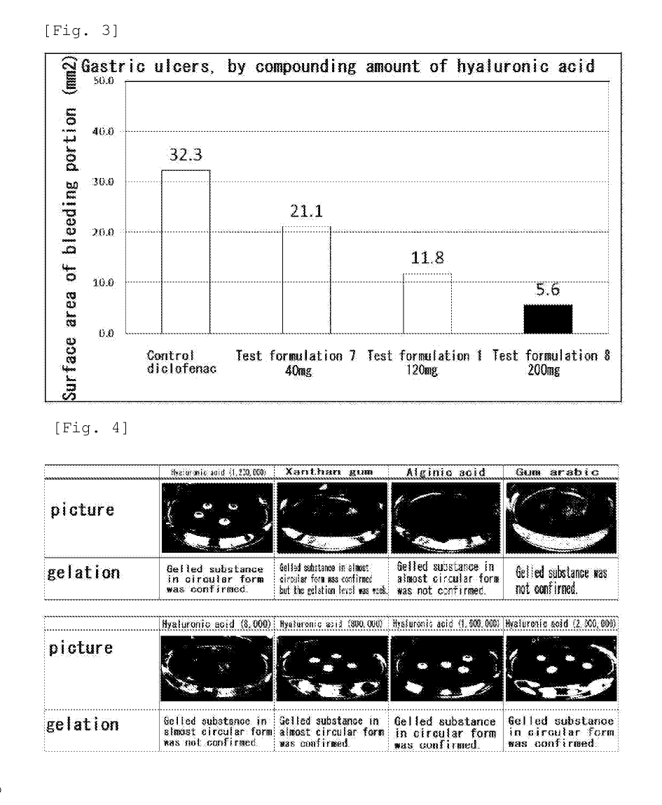

The result is shown in FIG. 4. In the cases of hyaluronic acid having molecular weights of 800,000, 1,200,000, 1,600,000 and 2,000,000, the formation of gelled substance in almost circular form was confirmed visually. In the case of xanthan gum, the formation of gelled substance similar to the cases of hyaluronic acid was also confirmed. Meanwhile, the gel in almost circular form was not formed in cases of alginic acid, gum arabic and hyaluronic acid having molecular weights of 8,000.

### EXAMPLE 1

Tablets

(1) diclofenac bulk 25 mg

(2) hyaluronic acid (mean molecular weight: 1,200,000) 15 mg

(3) lactose 174 mg

(4) corn starch 54 mg

(5) microcrystalline cellulose 10.5 mg

(6) magnesium stearate 1.5 mg

One tablet 280 mg

(1), (2), (3), ⅔ of (4) and ½ of (5) are sieved and mixed, followed by granulation. The remaining (4) and (5) are added to the granules and pressure-molded to prepare tablets.

### EXAMPLE 2

Tablets

(1) loxoprofen sodium hydrate bulk 68.1 mg (60 mg as anhydrate)

(2) hyaluronic acid (mean molecular weight: 1,200,000) 15 mg

(3) lactose 174 mg

(4) corn starch 54 mg

(5) microcrystalline cellulose 10.5 mg

(6) magnesium stearate 1.5 mg

One tablet 323.1 mg

(1), (2), (3), ⅔ of (4) and ½ of (5) are sieved and mixed, followed by granulation. The remaining (4) and (5) are added to the granules and pressure-molded to prepare tablets.

### EXAMPLE 3

Tablets

(1) acetylsalicylic acid bulk 100 mg

(2) hyaluronic acid (mean molecular weight: 1,200,000) 15 mg

(3) lactose 174 mg

(4) corn starch 54 mg

(5) microcrystalline cellulose 10.5 mg

(6) magnesium stearate 1.5 mg

One tablet 355 mg

(1), (2), (3), ⅔ of (4) and ½ of (5) are sieved and mixed, followed by granulation. The remaining (4) and (5) are added to the granules and pressure-molded to prepare tablets.

### EXAMPLE 4

Composition of Oral Liquid Medicine

(1) acetylsalicylic acid bulk 330 mg

(2) hyaluronic acid (mean molecular weight: 1,200,000) 45 mg

(3) purified sucrose 100 mg

(4) D-sorbitol 20 mg

(5) polyethyleneglycol 20 mg

(6) purified water proper amount

Total amount 5 mL

Oral Liquid Medicine containing acetylsalicylic acid 330 mg per 5 mL is prepared by mixing (1), (2), (3), (4), (5) and (6).

### EXAMPLE 5

Capsules

Composition of drug solution for capsule

(1) indomethacin bulk 25 mg

(2) hyaluronic acid (mean molecular weight: 1,200,000) 15 mg

(3) D-sorbitol 300 mg

(4) polyethyleneglycol 60 mg

Composition of coating film

(5) gelatin 100 mg

(6) concentrated glycerin 30 mg

(7) methyl p-hydroxybenzoate 0.2 mg

(8) propyl p-hydroxybenzoate 0.05 mg

(9) purified water proper amount

After mixing the drug solution of (1), (2), (3) and (4), a soft capsule containing acetylsalicylic acid 100 mg per one capsule is prepared with gelatin sheet produced by using coating film composition of (5), (6), (7), (8) and (9) according to a rotary method. In addition, the amount of drug solution per one capsule is 40 mg. The concentration (w/v) of hyaluronic acid is 5.26%.

1. A method for suppressing onset of gastric ulcer caused by a drug having an adverse effect of gastric ulcer, by using said drug in combination with a high-molecular polysaccharide having molecular weight of not less than 800,000 and not more than 3,000,000, which produces a gel when a 0.3% (w/v) aqueous solution thereof is added dropwise to artificial gastric juice.

2. A method for suppressing onset of gastric ulcer caused by a drug having an adverse effect of gastric ulcer, by incorporating a high-molecular polysaccharide having molecular weight of not less than 800,000 and not more than 3,000,000, which produces a gel when a 0.3% (w/v) aqueous solution thereof is added dropwise to artificial gastric juice, into a pharmaceutical composition containing said drug.

3. The method for suppressing onset of gastric ulcer according to claim 1, wherein the drug having an adverse effect of gastric ulcer is an anti-inflammatory drug (NSAIDs), a hormone preparation, an anticancer agent, a therapeutic agent of osteoporosis or an antiplatelet drug.

4. The method for suppressing onset of gastric ulcer according to claim 1, wherein the high-molecular polysaccharide is hyaluronic acid and/or a salt thereof, or Xanthan gum.

5. The method for suppressing onset of gastric ulcer according to claim 1, wherein the onset of gastric ulcer is suppressed without changing the approved administration and dosage of the drug having an adverse effect of gastric ulcer.

6. An oral pharmaceutical composition with suppressed onset of gastric ulcer which contains
a drug having an adverse effect of gastric ulcer,
a high-molecular polysaccharide having molecular weight of not less than 800,000 and not more than 3,000,000, which produces a gel when a 0.3% (w/v) aqueous solution thereof is added dropwise to artificial gastric juice, and
a pharmaceutically acceptable carrier.

7. The oral pharmaceutical composition according to claim 6, wherein the drug having an adverse effect of gastric ulcer is an anti-inflammatory drug (NSAIDs), a hormone preparation, an anticancer agent, a therapeutic agent of osteoporosis or an antiplatelet drug.

8. The oral pharmaceutical composition according to claim 6, wherein the high-molecular polysaccharide is hyaluronic acid and/or a salt thereof, or Xanthan gum.

9. A method for producing a pharmaceutical preparation with suppressed onset of gastric ulcer as an adverse effect, characterized by mixing previously a high-molecular polysaccharide having molecular weight of not less than 800,000 and not more than 3,000,000, which produces a gel when a 0.3% (w/v) aqueous solution thereof is added dropwise to artificial gastric juice, in a drug having an adverse effect of gastric ulcer, and formulating the mixture into a preparation.

10. The method for suppressing onset of gastric ulcer according to claim 2, wherein the drug having an adverse effect of gastric ulcer is an anti-inflammatory drug (NSAIDs), a hormone preparation, an anticancer agent, a therapeutic agent of osteoporosis or an antiplatelet drug.

11. The method for suppressing onset of gastric ulcer according to claim 2, wherein the high-molecular polysaccharide is hyaluronic acid and/or a salt thereof, or Xanthan gum.

12. The method for suppressing onset of gastric ulcer according to claim 2, wherein the onset of gastric ulcer is suppressed without changing the approved administration and dosage of the drug having an adverse effect of gastric ulcer.
